(19) United States
(12) Patent Application Publication
(10) Pub. No.: US 2007/0292523 A1
(43) Pub. Date: Dec. 20, 2007

(54) Dihydropyrimidine Formulations

(76) Inventors: Joey Moodley, Athlone (IE); Ivan Coulter, Dublin (IE)

Correspondence Address: JACOBSON HOLMAN PLLC, 400 SEVENTH STREET N.W., SUITE 600, WASHINGTON, DC, 20004, US

(21) Appl. No.: 11/663,836

(22) PCT Filed: Sep. 27, 2005
(86) PCT No.: PCT/IE05/00105

§ 371 Date: Mar. 27, 2007

Related U.S. Application Data
(60) Provisional application No. 60/612,784, filed on Sep. 27, 2004.
(60) Provisional application No. 60/612,785, filed on Sep. 27, 2004.
(60) Provisional application No. 60/612,786, filed on Sep. 27, 2004.

(51) Int. Cl. A61K 9/50; A61K 31/4422; A61K 31/513; A61K 31/55; A61K 31/56; A61K 36/16

(52) U.S. Cl. 424/497; 424/490; 424/752; 514/171; 514/215; 514/217; 514/256; 514/356

(57) ABSTRACT

A pharmaceutical formulation comprises a plurality of seamless minicapsules having a diameter of from 0.5 mm to 5 mm, the minicapsules having an encapsulating medium, and the mincapsules containing a dihydropyrimidine such as Nimodipine as an active ingredient.

### FIELD OF THE INVENTION

This invention relates to a dosage form of a dihydropyrimidine such as nimodipine.

### BACKGROUND OF THE INVENTION

Nimodipine belongs to the class of pharmacological agents known as calcium channel blockers. The contractile processes of smooth muscle cells are dependent upon calcium ions, which enter these cells during depolarisation as slow ionic transmembrane currents. Nimodipine inhibits calcium ion transfer into these cells and thus inhibits contractions of vascular smooth muscle. Nimodipine is a yellow crystalline substance, practically insoluble in water. Nimodipine is typically formulated as soft gelatin capsule for oral administration. Nimodipine is indicated for the improvement of neurological outcome by reducing the incidence and severity of ischemic deficits in patients with subarachnoid hemorrhage from ruptured intracranial berry aneurysms regardless of their post-ictus neurological condition. The precise mode of action is not clear.

Nimodipine is a poorly water soluble drug. Drugs that are poorly water soluble must be formulated in a way that improves their solubility and hence their bioavailability. In general, a drug that is in solution or suspension when administered by the oral route is rapidly and frequently instantaneously absorbed from the gastrointestinal tract resulting in a fast therapeutic action. However in many cases it is desirable to control the rate of absorption following oral administration in order to achieve the desired plasma profile or prolongation of action of the drug.

Furthermore, there is a need for a formulation which will allow practitioners to more easily administer nimodipine formulations especially to traumatised patients within the ICU environment. In the ICU context, prior to administration nimodipine formulations may be mixed with soft foods or liquids and administered to patients via tubing directly to the stomach or small intestine.

### STATEMENTS OF INVENTION

According to the invention there is provided a pharmaceutical formulation comprising a plurality of seamless minicapsules having a diameter of from 0.5 mm to 5 mm, the minicapsules having an encapsulating medium, and the mincapsules containing a dihydropyrimidine as an active ingredient.

In one embodiment the active ingredient is dispersed in the encapsulating medium.

In one embodiment at least some of the minicapsules comprise a core containing the active ingredient. In at least some of the minicapsules the active ingredient in the core may be solubilised in a pharmaceutically acceptable solvent and/or in a liquid phase. In at least some of the minicapsules the active ingredient may be in a solid form and/or in a semi-solid form.

The minicapsules may have a diameter of from 0.5 mm to 3.0 mm, from 1.2 mm to 2.0 mm, 1.4 mm to 1.8 mm.

In one embodiment at least some of the minicapsules have at least one coating to control the time and/or location of the release of the active entity. At least one coating may be an immediate release coating. At least one coating may be a sustained release coating. The coating may comprise a sustained release and an immediate release coating. At least one coating may be an enteric coating.

In one embodiment the rate-controlling coating is of a polymeric material. The rate-controlling coating may comprise amino methacrylate polymeric material.

The rate-controlling coating may be an acrylate and/or methacrylate copolymer with quaternary ammonium.

In one embodiment the coating comprises two copolymers, one of which is highly permeable and the other of which is poorly permeable. The weight ratio of highly permeable polymer to poorly permeable polymer may be from 5:95 to 15:85. The ratio may be from 10:90 to 15:85.

In one case the highly permeable copolymer is applied in the form of a polymer solution. The highly permeable copolymer may be insoluble in water. In one case the poorly permeable copolymer is applied in the form of a polymer solution. The poorly permeable copolymer may be insoluble in water.

In one embodiment the rate-controlling polymer coating contains methacrylate copolymer in the following ratio's 5:95; 10:90; 15:85 (w/w) as a mixture of Eudragit RL:Eudragit RS.

The copolymer mixture may comprise Eudragit RL 30D:Eudragit RS 30D. The copolymer mixture may comprise Eudragit RL 12.5:Eudragit RS12.5.

The coating comprises an enteric coating of a methacrylate polymer. The enteric coating may comprise Eudragit S 12.5 or Eudragit S100 providing 0 drug release in the stomach for up to 4 hours.

In one case at least some of the minicapsules are coated with an immediate release coating. The immediate release coating may be applied to a rate controlling coating. In one embodiment the immediate release coating contains a pharmaceutically active ingredient. An immediate release pharmaceutically active ingredient solution may be applied to a rate-controlling coating.

In one embodiment the active pharmaceutical ingredient is suspended or dissolved in the encapsulating medium of the seamless minicapsule. The encapsulating medium may be of one or more of gelatine, agar, a polyethylene glycol, starch, casein, chitosan, soya bean protein, safflower protein, alginates, gellan gum, carrageenan, xanthan gum, phtalated gelatine, succinated gelatine, cellulosephtalate-acetate, oleoresin, polyvinylacetate, hydroxypropyl methyl cellulose, polymerisates of acrylic or methacrylic esters, polyvinylacetate-phtalate and combinations thereof.

In one case the active ingredient is suspended or dissolved in the encapsulating medium. The active ingredient may be micronised or nanonised. In this case the active ingredient may have a particle size of less than 100 microns.

In another embodiment at least some of the minicapsules comprise a core containing micronised or nanonised active ingredient and the encapsulating medium contains micronised or nanonised active pharmaceutical ingredient suspended or dissolved in the encapsulating medium to enhance the potency of the seamless minicapsules.

A permeability enhancing agent may be suspended or dissolved in the encapsulating medium to enhance active bioavailability.

In one embodiment the formulation comprises a buffer layer.

In another embodiment at least some of the minicapsules are provided with or contain a bioadhesive, typically a mucoadhesive.

In one case the bioadhesive comprises from 0% to 10% by weight of one or more of the following polymer classes:—polyacrylates; polyanhydrides; chitosans; carbopols; cellulose; methylcellulose; methylated deoxycellulose (m-doc™); lectins.

The bioadhesive may comprise from 0% to 10% by weight of one or more of the following thiolated or otherwise derivatised polymers:—polyacrylates; polyanhydrides; chitosans; carbopols; cellulose; methylcellulose; methylated deoxycellulose (m-doc™); lectins.

In one case the bioadhesive comprises a coating. Alternatively or additionally the bioadhesive is incorporated into a part or layer of the minicapsule such as rate-controlling layer and/or the encapsulating medium.

In one embodiment at least some of the minicapsules have a layer such as an outer layer which is divided into at least two parts. The parts may have the same or different compositions.

In one embodiment the formulation comprises at least two populations of sustained release minicapsules. The populations may have different in-vitro dissolution profiles.

In another aspect the invention provides a formulation of a dihydropyrimidine as the active ingredient comprising a plurality of seamless minicapsules having at least two populations selected from:—
a first minicapsule population in which the minicapsules comprise a core containing the active ingredient and an encapsulating medium, the minicapsules having a diameter of from 0.5 mm to 5 mm;
a second minicapsule population in which the minicapsules comprise a plurality of particles containing the active entity dispersed in an encapsulating medium, the minicapsules having a diameter of from 0.5 mm to 5 mm; and
a third micro or mini particles population in which the minicapsules comprise an inert core and at least one layer around the core, the layer containing the active ingredient.

In one embodiment the dihydropyridine is nimodipine.

The dihydropyridine may be selected from felodipine, nicardipine nifedipine, istradipine, amlodipine and nisoldipine.

In one case at least some of the minicapsules comprise a core containing nimodipine. The formulation may comprise an immediate release coating which contains nimodipine.

In one embodiment the coating comprises a rate-controlling coating to achieve therapeutically effective plasma levels of the active ingredient over at least 12 hours in a human patient.

The coating may comprise a rate-controlling coating to achieve therapeutically effective plasma levels of the active ingredient over at least 24 hours in a human patient.

In one case the formulation provides a dissolution profile in a pre-determined media such that NMT 25% of the solubilised pharmaceutical active ingredient is released after 1 hour, NMT 40% after 4 hours, NMT 70% after 8 hours and 75 to 100% after 12 hours.

In another case the formulation provides a dissolution profile in predetermined media such that 10 to 15% of the solubilised pharmaceutical active ingredient is released after 1 hour, about 15 to 30% is released after 4 hours, about 35 to 50% is released after 9 hours, about 45 to 65% is released after 12 hours and at least 80% is released after 24 hours.

In one embodiment an enteric coated minicapsule is combined with two sustained release coated minicapsules to provide a pulsed release dissolution profile.

In one case greater than 80% (w/w by potency) of the formulation is comprised of sustained release minicapsules.

In one embodiment the minicapsules provide extended residence times in the small intestine for a period of at least 5 hours, preferably at least 7 hours and more preferably in the 8-24 hours range to enable maximal bioactivity of the core active, locally or systemically.

The minicapsules may provide extended residence times in the nasal passage to enable maximal bioactivity of the core active agent, locally or systemically.

The minicapsules may provide extended residence times in the rectal passage to enable maximal bioactivity of the core active agent, locally or systemically.

The minicapsules may be capable of extended residence times in the vagina or intrauterine to enable maximal bioactivity of the core agent, locally or systemically.

In one embodiment the minicapsules are filled into hard gelatin capsules.

The minicapsules may be filled into a sachet.

The minicapsules may be suspended in oil as a lubricant.

The minicapsules may be contained within a wide gauge syringe that is compatible with tube delivery.

The minicapsules may be in the form of a sprinkle.

The minicapsules may be formulated as a suppository for rectal or vaginal or intrauterine administration.

The minicapsules may be formulated for nasal administration.

In one embodiment the formulation contains at least one further active entity. The further active entity may be a P-gp/P450 inhibitor. The further active entity may be carbamazepine, valproic acid, cimetidine or a tryptan such as sumatriptan. The formulation may be for treatment of Alzheimers disease wherein the further active entity comprises a cholinesterase inhibitor (such as donepezil, rivastigmine, galantamine) and one or more from the following classes: vitamins, statins, estrogen, nootrophic agents, ginkgo biloba, anti-inflammatory agents, anti-depressants, anti-psychotics, and mood stabilizers.

The further active entity may be selected from one or more of a statin, a thiazidediuretic, a beta blocker, an ACE inhibitor, folic acid, co-enzyme Q10, and an anticoagulant.

In one case the further active entity is present in a seamless minicapsule. The further active entity may be present in at least some of the seamless minicapsules.

The invention also provides a formulation comprising a capsule containing a plurality of minicapsules of the invention.

The capsule may contain another entity.

The other entity may be in a powder, liquid, solid, semi-solid or gaseous form.

The other entity may be an active entity.

In another embodiment the formulation comprises a tablet or pellet containing a plurality of minicapsules. The tablet or pellet may contain another entity. The other entity may be an active entity.

The invention provides a controlled release technology which will allow the delivery of a dihydropyrimidine in solution to the optimum site of absorption/action in the gastrointestinal tract.

The dihydropyrimidine formulations of the invention will allow practitioners to more easily administer active pharmaceutical formulations especially to traumatised patients within the ICU environment. In the ICU context, prior to administration of the formulations may be mixed with soft foods or liquids and administered to patients via tubing directly to the stomach or small intestine.

In one embodiment the rate-controlling polymer coat contains Methacrylate Copolymer as described in USP/NF in the following ratio's 5:95; 10:90; 15:85 as a mixture of Eudragit RL: Eudragit RS more especially Eudragit RL 12.5:Eudragit RS 12.5 or Eudragit RL30D: Eudragit RS30D or Eudragit E100 or Eudragit E PO or a combination thereof.

In another embodiment the rate-controlling polymer coat is an Enteric Coat of Eudragit S 12.5 or Eudragit L100 or Eudragit S100 or Eudragit 30D or a combination thereof providing 0 drug release in the stomach for up to 4 hours.

In a further embodiment an enteric coated pharmaceutical active ingredient minicapsule is combined with two SR coated pharmaceutical active ingredient minicapsules components to give Pulsed Release dissolution profile.

### BRIEF DESCRIPTION OF THE DRAWINGS

The invention will be more clearly understood from the following description thereof given by way of example only with reference to the accompanying drawings, in which:—

FIGS. 1 to 3 are graphs representing nimodipine multiparticulate seamless minicapsule dissolution profiles;

FIG. 1: Example 1 (Batch MY11) Nimodipine Multiparticulate Minicapsule—Immediate Release Dissolution Data (IR)

FIG. 2: Example 2 (Batch MY21) Nimodipine Multiparticulate Minicapsule—Sustained/Controlled Release Dissolution Data (SR/CR)

FIG. 3: Example 3 (Batch MY22) Nimodipine Multiparticulate Minicapsule—Sustained/Controlled Release Dissolution Data (SR/CR).

FIGS. 4 to 10 are graphs which illustrate the impact of different release rates of nimodipine on % percent release—first order release;

FIG. 4: Percent Release versus Time Profile—First Order Release

FIG. 5: Simulated Single Dose Plasma Concentration versus Time Profiles (0-4 h)—First Order Release (Dose=60 mg)

FIG. 6: Simulated Single Dose Plasma Concentration versus Time Profiles (0-4 h)—First Order Release (Dose=120 mg)

FIG. 7: Simulated Single Dose Plasma Concentration versus Time Profiles (0-4 h)—First Order Release (Dose=180 mg)

FIG. 8: Simulated Single Dose Plasma Concentration versus Time Profiles (0-4 h)—First Order Release (K01=0.7525; Dose=180 mg)

FIG. 9: Simulated Steady State Plasma Concentration versus Time Profiles (0-24 h)—First Order Release (K01=0.7525; Dose 180 mg BID and K01=5.018; 60 mg QID)

FIG. 10: Simulated Steady State Plasma Concentration versus Time Profiles (0-24 h)—First Order Release (K01=0.7525; Dose 180 mg BID and K01=5.018; Dose 90 mg QID)

### DETAILED DESCRIPTION

The invention will be more clearly understood from the following description thereof given by way of example only.

The invention provides a multiparticulate seamless minicapsule formulation of a dihydropyrimidine for twice or once daily administration to a patient, comprising sustained release particles each having a core containing a solubilised pharmaceutical active ingredient in a solvent or liquid phase as a seamless minicapsule, the core being coated with a rate-controlling polymer coat comprised of ammonia methacrylate copolymers in an amount sufficient to achieve therapeutically effective plasma levels of the active ingredient over at least 12 or 24 hours.

The pharmaceutical active seamless minicapsules were manufactured according to Freund Industrial Co, Ltd. U.S. Pat. No. 5,882,680 (Seamless Capsule and Method of Manufacturing the Same), the entire contents of which are incorporated herein by reference.

The principle of seamless minicapsule formation is the utilisation of “surface tension”, when two different solutions (which are not or hardly dissolved with each other) contact each other, which works by reducing the contact area of the two different solutions.

After encapsulating the core solution which is ejected through an orifice with a certain diameter, with the shell solution which is also ejected through an outer orifice, the encapsulated sphere is then ejected into a cooling or hardening solution and the outer shell solution is gelled or solidified. This briefly describes the formation of seamless minicapsules.

The core solution is mainly a hydrophobic solution or suspension. The outer shell solution is normally gelatin based. However a hydrophilic solution can also be encapsulated with the existence of an intermediate solution, which can avoid the direct contact of the hydrophilic core solution with the outer shell.

With the nozzle having a single orifice, a minicapsule or a bead of shell/core mixed suspension can be processed.

With the nozzle having two orifices (centre and outer), a hydrophobic solution can be encapsulated.

With the nozzle having three or more orifices seamless minicapsules for various applications can be processed. (Ref U.S. Pat. No. 5,882,680)

By using the above described manufacturing processing method as per U.S. Pat. No. 5,882,680 for multiparticulate seamless minicapsules, active pharmaceutical multiparticulate seamless minicapsules were produced. The completed seamless minicapsules preferably have an average diameter of 0.5-3.00 mm, more especially in the range 1.50-1.80 mm.

According to one embodiment a portion or all of the sustained release particles further comprise an immediate release coating applied onto the rate-controlling polymer coat, which immediate release coating comprising a solubilised pharmaceutical active ingredient in a liquid phase.

In an alternative embodiment, the formulation can contain a portion of immediate release minicapsules each comprising a core of solubilised active pharmaceutical ingredient in a liquid phase.

The formulation according to the invention may also comprise at least two populations of sustained release seamless minicapsules having two different in vitro dissolution profiles.

Also preferably, the formulation according to the invention provides a dissolution profile in a pre-selected media such that about NMT 25% of the solubilised active ingredient is released after 1 hour; NMT 40% after 4 hours; NMT 70% after 8 hours; 75 to 100% after 12 hours.

In an alternative embodiment the formulation provides a dissolution profile in a pre-determined media such that about 10 to 15% of the solubilised active ingredient is released after 1 hour; 15 to 30% is released after 4 hours; about 35 to 50% is release after 9 hours; about 45 to 65% is released after 12 hours and at least 80% is released after 24 hours.

Also, in a preferred embodiment greater than 80% (w/w by potency) of the formulation is comprised of sustained release seamless minicapsules.

In a preferred embodiment the rate-controlling polymer coat contains Ammonia Methacrylate Copolymer Type A and Ammonia Methacrylate Copolymer Type B as described in USP/NF.

Such copolymers are manufactured and marketed by Degussa (Rohm) GmbH, Darmstadt, Germany.

Most preferably the rate-controlling polymer coat contains a 5:95 or 10:90 or 15:85 mixture of Eudragit RL: Eudragit RS most especially Eudragit RL30D: Eudragit RS30D or Eudragit RL 12.5:Eudragit RS 12.5

Preferably the sustained release seamless minicapsules following application of the rate-controlling polymer coat are dried at a temperature of about 40-50 deg C., typically for up to 24 hours.

In a preferred embodiment the formulation is encapsulated, for example in a hard gelatin capsule.

The sustained release seamless minicapsules are formed by coating the active seamless minicapsule with the rate-controlling polymer coat comprised of ammonio methacrylate copolymers such as those sold under the Trade Mark EUDRAGIT.

EUDRAGIT polymers are polymeric lacquer substances based on acrylates and/or methacrylates. The polymeric materials sold under the Trade Mark EUDRAGIT RL and EUDRAGIT RS are acrylic resins comprising copolymers of acrylic and methacrylic acid esthers with a low content of quaternary ammonium groups and are described in the “EUDRAGIT” brochure of Messrs. Degussa (Rohm Pharma) GmbH wherein detailed physical-chemical data of these products are given. The ammonium groups are present as salts and give rise to the permeability of the lacquer films. EUDRAGIT RL is freely permeable or RS slightly permeable, independent of pH.

The rate-controlling polymer coat maybe built up by applying a plurality of coats of polymer solution or suspension to the minicapsule as hereafter described. The polymer solution or suspension contains the polymer(s) dissolved or suspended, respectively in a suitable aqueous or organic solvent or mixture of solvents, optionally in the presence of a lubricant. Suitable lubricants are talc, stearic acid, magnesium stearate and sodium stearate. A particularly preferred lubricant is talc.

The polymer solution or suspension may optionally include a plasticizing agent. Suitable plasticizing agents include polyethylene glycol, propyleneglycol, glycerol, triacetin, dimethyl phthalate.diethyl phthalate, dibutyl phthalate, dibutyl sebacate or varying percentages of acetylated monoglycerides.

Suitable organic solvents include isopropyl alcohol (IPA) or acetone or a mixture.

The polymer solution or suspension maybe applied to the minicapsules preferably using an automated system such as a GLATT fluidised bed processor, Vector Flow Coater System or an Aeromatic fluidised bed processor.

Polymer solution/suspension in the quantity of 5-75 ml per kilogram of minicapsules may be applied to the minicapsules using one of the listed automated fluidised bed processing systems to given target polymer coating weight.

In accordance with the invention the drug loaded minicapsules are coated with the rate-controlling polymers to achieve a target dissolution rate. The drug released from these minicapsules is diffusion controlled as the polymer swells and becomes permeable, it allows for the controlled release in the GIT. In order to achieve a suitable dissolution profile, the following parameters require consideration, efficient process/conditions, drug solubility/particle size, minicapsule surface area, minicapsule diameter and coating polymer suitability.

The mucoadhesive controlled GIT transit minicapsules are formed by coating the active seamless minicapsules with the transit-controlling polymer coat comprised of, for example various cellulose or cellulose derivatives such as chitosan or those sold under the brand name Carbopol.

The minicapsule gelatine shell can be modified to comprise a sphere having two hemispheres. Each hemisphere contains variable concentrations of gelatine alone or gelatine in combination with, for example, a mucoadhesive and/or an enteric material. This aspect of the invention will ensure that the active is both in close proximity with the intestinal lumen and protected from intestinal degradative attack.

Nimodipine is a dihydropyridine derivative and belongs to the class of pharmacological agents known as calcium channel blockers. The contractile processes of smooth muscle cells are dependent upon calcium ions, which enter these cells during depolarisation as slow ionic transmembrane currents. Nimodipine inhibits calcium ion transfer into these cells and thus inhibits contractions of vascular smooth muscle. Nimodipine is a yellow crystalline substance, practically insoluble in water. Nimodipine is typically formulated as soft gelatine capsules for oral administration. Nimodipine is indicated for the improvement of neurological outcome by reducing the incidence and severity of ischemic deficits in patients with subarachnoid haemorrhage from ruptured intracranial berry aneurysms regardless of their post-ictus neurological condition. The precise mode of action is not clear.

The invention provides an oral nimodipine multiparticulate seamless minicapsule formulation for twice or once daily administration to a patient, comprising sustained release particles each having a core containing a solubilised nimodipine in a solvent or liquid phase as a seamless minicapsule, the core being coated with a rate-controlling polymer coat comprised of ammonia methacrylate copolymers in an amount sufficient to achieve therapeutically effective plasma levels of nimodipine over at least 12 or 24 hours.

### EXAMPLE A

Core Solution

| | | |
|---|---|---|
| Nimodipine USP/EP | 200 | grams |
| PEG 400 | 800 | grams |

Median Solution

| | | |
|---|---|---|
| Vegetable or Mineral Oil | 1000 | grams |

Film Solution

| | | |
|---|---|---|
| Gelatin | 225 | grams |
| Sorbitol | 25 | grams |
| Purified Water | 750 | grams |

Polymer Coating Solution

| Eudragit RL | 5% | w/w |
|---|---|---|
| Eudragit RS | 95% | w/w |
| Talc | as required | |
| Minicapsule diameter | 1.50 | mm |

The Nimodipine Multiparticulate Seamless Minicapsules were manufactured according to Freund Industrial Co. Ltd U.S. Pat. No. 5,882,680 (Seamless Capsule and Method of Manufacturing Same) and as described in the Summary of the Invention Section.

In order to coat the core solubilised seamless minicapsules, a coating solution of 6.25% Eudragit RL (5% w/w) and Eudragit RS (95% w/w) dissolved in isopropyl alcohol/acetone mixture was sprayed onto the minicapsules using an automated fluidised bed processor. Talc was added simultaneously to avoid agglomeration.

The coated minicapsules were dried in an environmentally controlled drier for between 12 to 24 hours to remove any residual solvents

Encapsulation 10% immediate release/90% sustained release.

Nimodipine seamless minicapsules uncoated (10% w/w by potency) and the polymer coated minicapsules (90% w/w by potency) from the above were blended using a suitable mechanical blender.

The resultant blend was filled into suitable gelatin capsules to the required target strength.

### EXAMPLE B

Core Solution

| | | |
|---|---|---|
| Nimodipine USP/EP | 750 | grams |
| Gelatin | 1125 | grams |
| Sorbitol | 125 | grams |
| Purified Water | 4250 | grams |

Polymer Coating solution

| Eudragit RS | 85% | w/w |
|---|---|---|
| Eudragit RL | 5% | w/w |
| Dibutyl Sebacate | 10% | w/w |
| Talc | as required | |
| Minicapsule Diameter | 1.50 | mm |

The above seamless minicapsules were manufactured in the same way as Example A with the following exceptions:—
1. The core solution was treated with a High Pressure Homogeniser. The median and film solutions were excluded from this example.
2. The polymer solution included a 10% plasticiser. The Eudragit RS/RL were adjusted proportionately.
3. Two sustained release components were used. SR component 1 was as used in Example A. SR component 2 was what was prepared in Example B which included the plasticiser in the polymer coating solution.

The Nimodipine minicapsule uncoated (10-20% w/w by potency), SR 1 (40-45% w/w by potency), SR 2 (40-45% w/w by potency) were blended as per Example A and filled into gelatin capsules to the target strength.

### EXAMPLE C

Core Solution

| | | |
|---|---|---|
| Nimodipine USP/EP | 500 | grams |
| Low Viscosity MCT | 500 | grams |

Film Solution

| | | |
|---|---|---|
| Gelatin | 590 | grams |
| Sorbitol | 70 | grams |
| Nimodipine USP/EP | 340 | grams |
| Purified Water | 2290 | grams |

Polymer Coating Solution

| Eudragit S | | |
|---|---|---|
| Isopropyl Alcohol/acetone | as required | |
| Talc | as required | |
| Minicapsule Diameter | 1.50 | mm |

The above seamless minicapsules were manufactured in the same way as Example A with the following exceptions:—
1. The core solution was pre-treated with an Ultra Centrifugal Mill.
2. The film solution Nimodipine, was pre-treated with a High Pressure Homogeniser
3. The median solution was excluded this formulation.
4. Eudragit S was used as the polymer coat to provide an enteric coat with 0 drug release of up to 4 hours to the minicapsules, to target the drug release to the GIT and providing a pulsed release profile.

A percentage of the Enteric Coated Nimodipine minicapsules and a percentage of the coated minicapsules from Example A (as required) and a percentage of the uncoated minicapsules from Example A (as required) were blended as per in Example A and filled into suitable gelatin capsules to the target strength.

### EXAMPLE 1

Core Solution

| | |
|---|---|
| Micronised Nimodipine USP/EP | 11.7% |
| PEG 400 | 46.6% |

Median Solution

| | |
|---|---|
| Medium-Chain Triglycerides (MCT) | 2.4% |
| Sucrose Acetate Isobutylate (SAIB) | 9.4% |

Film Solution

| | |
|---|---|
| Gelatin | 30% |
| Purified Water | as required |
| Minicapsule diameter | 1.50-1.80 mm |

The Immediate Release (IR) Nimodipine Multiparticulate Seamless Minicapsules were manufactured according to Freund Industrial Co. Ltd U.S. Pat. No. 5,882,680 (Seamless Capsule and Method of Manufacturing Same) and as described in the Summary of the Invention Section. The multiparticulate minicapsules produced in this example achieved an Immediate Release Dissolution Profile as follows.

Dissolution Method

Apparatus: Vankel VK7025 fully auto mated with Cary Win UV

Dissolution Medium Gastric Juice with 1% SDS pH 1.2 (900 mls)

Stirring: USP Apparatus 2 (Paddles) at 100 rpm

UV: 330 nm

Dissolution Profile of Nimodipine Multiparticulate Immediate Release Seamless Minicapsules Batch MY11

| | Time | | | | | | | | | | | | | | | | |
|---|---|---|---|---|---|---|---|---|---|---|---|---|---|---|---|---|---|
| | (Mins) | | | | | | | | | | | | | | | | |
| | 0 | 15 | 30 | 45 | 60 | 75 | 90 | 105 | 120 | 135 | 150 | 165 | 180 | 195 | 210 | 225 | 240 |
| Batch | 0 | 8 | 22 | 34 | 44 | 52 | 58 | 64 | 69 | 74 | 78 | 82 | 85 | 89 | 92 | 95 | 99 |
| MY11 % | | | | | | | | | | | | | | | | | |
| Released | | | | | | | | | | | | | | | | | |

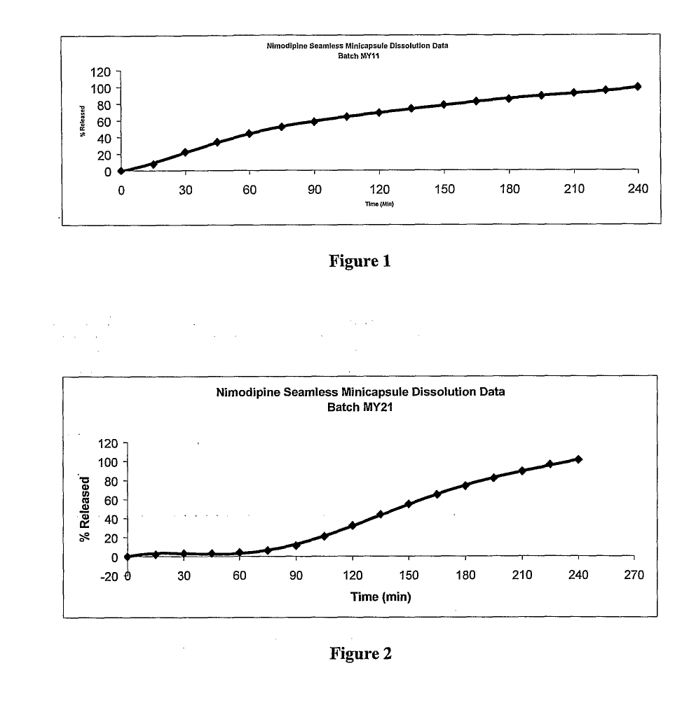

This data is graphically presented in FIG. 1.

The immediate release product was then filled into hard gelatine capsules to the required dosage strength. Furthermore the invention allows for the immediate release product to be produced in combination with a Sustained Release or Controlled Release multiparticulate minicapsule product in varying ratios of IR:SR/CR. The immediate release minicapsules can be combined with a Sustained or Controlled release minicapsule component in the following ratio's (w/w by potency) e.g. 10% Immediate Release (IR)+90% Sustained (SR)/Controlled Release (CR) minicapsules; 20% IR+80% SR/CR; 30% IR+70% SR/CR; 40% IR+60% SR/CR and 50% IR+50% SR/CR.

### EXAMPLE 2

Core Solution

| | |
|---|---|
| Micronised Nimodipine USP/EP | 11.7% |
| PEG 400 | 46.6% |

Median Solution

| | |
|---|---|
| MCT | 2.4% |
| SAIB | 9.4% |

Film Solution

| | |
|---|---|
| Gelatin | 20.2% |
| Sorbitol | 3.0% |
| Hydroxypropylmethyl Cellulose Phthlate (HP55) | 6.1% |
| Sodium Hydroxide (NaOH) | 0.7% |

The above Example 2 were manufactured according to Freund Industrial Co. Ltd U.S. Pat. No. 5,882,680 (Seamless Capsule and Method of Manufacturing Same).

In order to control the release (SR) of the Nimodipine over an extended period of time, Hydroxypropylmethyl Cellose Phthalate (HP55) was added to the Film Solution to act as a retarding agent which controlled the release of the Nimodipine over a given period. The multiparticulate minicapsules produced in this example achieved a Sustained/Controlled Release Dissolution Profile as follows.

Dissolution Method

Apparatus: Vankel VK7025 fully auto mated with Cary Win UV

Dissolution Medium: Gastric Juice with 1% SDS pH 1.2 (900 mls)

Stirring: USP Apparatus 2 (Paddles) at 100 rpm

UV: 330 nm

Dissolution Profile of Nimodipine Multiparticulate Sustained Release Seamless Minicapsules Batch MY21

| | Time | | | | | | | | | | | | | | | | |
|---|---|---|---|---|---|---|---|---|---|---|---|---|---|---|---|---|---|
| | (Mins) | | | | | | | | | | | | | | | | |
| | 0 | 15 | 30 | 45 | 60 | 75 | 90 | 105 | 120 | 135 | 150 | 165 | 180 | 195 | 210 | 225 | 240 |
| Batch | 0 | 2 | 3 | 3 | 4 | 6 | 11 | 21 | 32 | 44 | 55 | 65 | 74 | 82 | 89 | 96 | 101 |
| MY21 % | | | | | | | | | | | | | | | | | |
| Released | | | | | | | | | | | | | | | | | |

This data is graphically presented in FIG. 2.

The resultant multiparticulate minicapsules were filled into suitable hard gelatin capsules to the required target strength, typically 30/60/90/120 or 180 mg. Furthermore the invention allows for the combination of the SR/CR multiparticulate minicapsule with an immediate release multiparticulate minicapsule in varying ratio's of SR/CR: IR as stated in the claims (% percent Example 2+1). The IR+SR/CR combination ratio's are as per Example 1.

### EXAMPLE 3

Core Solution

| | |
|---|---|
| Micronised Nimodipine USP/EP | 37.5% |
| Gelatin | 56.3% |
| Sorbitol | 6.3% |
| Purified Water | as required |

Polymer Coating solution

| Eudragit RS | 85% | w/w |
|---|---|---|
| Eudragit RL | 5% | w/w |
| Dibutyl Sebacate | 10% | w/w |
| Talc | as required | |
| Minicapsule Diameter | 1.50 1.80 | mm |

The above seamless minicapsules were manufactured in the same way as Example 1 &2 with the following exceptions:—
4. The core solution was treated with a High Pressure Homogeniser.
5. The median and film solutions of examples 1 and 2 were excluded from this example.
6. The polymer coating solution included a 10% plasticiser. The Eudragit RS/RL were adjusted proportionately.

The process used to manufacture the multiparticulate minicapsules in this example in principle was the same as used in Example 1 & 2 with the exception that only a single orifice dosing system was used instead of the normal multiple dosing orifice system. By using a single dosing orifice a uniform solid gelatine pellet or sphere is produced to a specified particle size. This method produces a durable sphere in a gelatine format that includes the active ingredient which in turn allows the sphere or multipaticulate pellet to be further processes with various polymer coating systems. The multiparticulate minicapsules produced in this example achieved a Sustained/Controlled Release Dissolution Profile as follows.

Dissolution Method

Apparatus: Vankel VK7025 fully auto mated with Cary Win UV

Dissolution Medium Gastric Juice with 1% SDS pH 1.2 (900 mls)

Stirring: USP Apparatus 2 (Paddles) at 100 rpm

UV: 330 nm

Dissolution Profile of Nimodipine Multiparticulate Sustained Release Seamless Minicapsules Batch MY22

| | Time | | | | | | | | | | | | | | | | |
|---|---|---|---|---|---|---|---|---|---|---|---|---|---|---|---|---|---|
| | (Mins) | | | | | | | | | | | | | | | | |
| | 0 | 15 | 30 | 45 | 60 | 75 | 90 | 105 | 120 | 135 | 150 | 165 | 180 | 195 | 210 | 225 | 240 |
| Batch | 0 | 2 | 3 | 3 | 4 | 5 | 6 | 8 | 10 | 12 | 15 | 19 | 22 | 26 | 31 | 36 | 41 |
| MY22 % | | | | | | | | | | | | | | | | | |
| Released | | | | | | | | | | | | | | | | | |

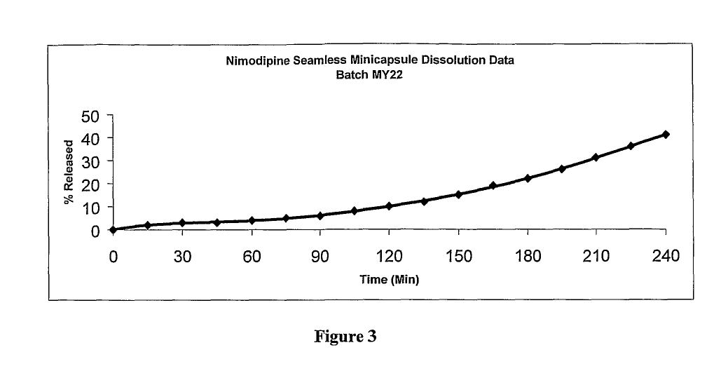

This data is graphically presented in FIG. 3.

Furthermore the invention allows for the combination of a SR/CR multiparticulate minicapsule with another SR/CR multiparticulate minicapsule and a IR multiparticulate minicapsule or other combinations thereof in varying ratio's of SR/CR:SR/CR:IR as stated in the claims (% percent Example 2+3+1).

A population of minicapsules from Example 2, Example 3 and Example 1 in varying ratio's as stated herein below were removed and blended in a suitable mechanical blender. The blended components were then filled into hard gelatine capsule to the required target strength.

Example 2 (45%)+Example 3 (45%)+Example 1 (10%)

Example 2 (50%)+Example 3 (30%)+Example 1 (20%)

Example 2 (30%)+Example 3 (60%)+Example 1 (10%)

Drug Layering

A multiparticulate drug layering process or technique may be used to compliment in combination with the invention. This process or technique is an art that is widely used and is accessible to a variety of formulators in the drug delivery arena.

This technique is known to be used by a number of companies in their technologies namely Eurand in their DIFFUCAP Technology, Shire—MICROTROL Technology, KV Pharmaceuticals—KV/24 Technology, Elan—SODAS Technology, to name a few.

The layering process involves applying an active ingredient and/or excipients onto an inert core e.g. non-pareils using a coating pan or fluid bed coater with a solution/suspension. In some instances the solution/suspension can contain both active ingredient and the binder which is then sprayed onto the inert cores. The other method of layering is the active is directly applied in a powder form by gravity or by auger feeder and adhesion to the inert cores is ensured by spraying a liquid binder onto the inert cores.

A further layering method is the inert core is substituted with a active sphere or granule with a particle size in the range of 0.5-1.5 mm and the layering process is carried out by spraying or dry powder layering as described above.

WO 95/14460 and WO 96/01621 are examples that describe different layering processes.

Multiparticulate layering processes using a spherical inert core such as non-pareils in most instances produce a homogeneous drug loaded particle with a spherical shape. These spherical shaped particles in turn lend themselves favourably to coating with various polymers to provide a desired drug release profile.

Multiparticulate layered spheres produced here can be used in combination with the current invention to achieve the desired dissolution profile for a specific product.

### EXAMPLE 4

| Micronised Nimodipine USP/EP | 500 | grams |
|---|---|---|
| Fumaric Acid | 0-125 | grams |
| Citric Acid | 0-125 | grams |
| Talc | 5-250 | grams |
| Sodium Lauryl Sulphate | 0.125 | grams |
| Sugar spheres (Non-Pareils) | 250 | grams |
| Kollidon 30 (Povidone) | 50-150 | grams |
| Eudragit RL | 5-15 | grams |
| Eudragit RS | 35-50 | grams |
| Isopropyl Alcohol | as required | |
| Acetone | as required | |
| Diameter Multiparticulate Spheres | 1.50-1.80 | mm |

The above example was produced by the multiparticulate layering process. This drug layering process is a well known and widely used technique in the drug delivery industry and is regularly used by formulation scientist to develop new delivery systems. The Nimodipine Applied Beads (IR) were manufactured as follows.

Nimodipine, Fumaric Acid or Citric Acid or both, talc and sodium lauryl sulphate (active blend) were blended in a suitable Y-Cone blender. The active blend was applied using a suitable fluid bed system onto non-pareils using a suitable binder or adhering solution, such as Povidone from a suitable organic or aqueous solution such as isopropyl alcohol. The resultant immediate release beads were dried for approx 24 hours. The dried multiparticulate spheres were then screened and the appropriate fractions retained.

The applied beads (IR) were then further processed. A coating solution of a 6.25% solution of Eudragit RS (75-95% w/w) and Eudragit RL (5-25% w/w) dissolved in isopropyl alcohol/acetone mix was sprayed onto the applied beads using a suitable fluid bed system. Talc was added simultaneously via a mechanical feeder to prevent agglomeration. The result was a layered applied sphere with a rate-controlling polymer having a pre-determined dissolution profile.

The resultant coated spheres (SR) from this example were then blended with a percentage of the applied (IR) spheres. The blended spheres from the above were filled into hard gelatine capsules to a target strength.

Furthermore the above example could also be combined with other the examples listed above. The following combinations in varying % percent ratio's can also be produced to give a pre-determined dissolution profile:—

Example 1+2+3+4 or Example 2+3+4 or Example 3+4 and the like. The following ratios are listed below as examples of the varying combinations that can be produced by removing a partial population of minicapsules from each of the above examples.

Example 1 (10%)+Example 2 (30%)+Example 3 (30%)+Example 4 (30%)

Example 2 (25%)+Example 3 (25%)+Example 4 (50%)

Example 3 (50%)+Example 4 (50%)

### EXAMPLE 5

Core Solution

| | | |
|---|---|---|
| Nimodipine USP/BP | 500 | grams |
| Low Viscosity MCT | 500 | grams |

Film Solution

| | | |
|---|---|---|
| Gelatin | 590 | grams |
| Sorbitol | 70 | grams |
| Nimodipine USP/EP | 340 | grams |
| Purified Water | 2290 | grams |

Polymer Coating Solution

| Eudragit S | as required | |
|---|---|---|
| Isopropyl Alcohol/acetone | as required | |
| Talc | as required | |
| Minicapsule Diameter | 1.50-1.80 | mm |

The above seamless minicapsules were manufactured in the same way as Example 1 with the following exceptions:—
5. The core solution was pre-treated with an Ultra Centrifugal Mill.
6. The film solution Nimodipine, was pre-treated with a High Pressure Homogeniser
7. The median solution was excluded this formulation.
8. Eudragit S was used as the polymer coat to provide an enteric coat with 0 drug release of up to 4 hours to the minicapsules, to target the drug release to the GIT and providing a pulsed release profile.

A percentage of the Enteric Coated Nimodipine minicapsules and a percentage of the coated minicapsules from Example 1 (as required) and a percentage of the uncoated minicapsules from Example 1(as required) were blended as per in Example 1 and filled into suitable gelatin capsules to the target strength.

### EXAMPLE 6

Core Solution

| | | |
|---|---|---|
| Nifedipine USP/EP | 100-400 | grams |
| PEG 400 | 400-800 | grams |

Median Solution

| | | |
|---|---|---|
| Low Viscosity MCT | 500-1000 | grams |

Film Solution

| | | |
|---|---|---|
| Gelatin | 590 | grams |
| Sorbitol | 70 | grams |
| Nifedipine USP/EP | 100-400 | grams |
| Purified Water | 1000-2500 | grams |

Polymer Coating Solution

| Eudragir S | as required | |
|---|---|---|
| Isopropyl Alcohol/Acetone | as required | |
| Talc | as required | |
| Minicapsule Diameter | 1.50-1.80 | mm |

The above seamless minicapsules were manufactured in the same way as Example 1 with the following exceptions:—

1. The Nifedipine core solution was pre-treated with an Ultra Centrifugal Mill.

2. The Nifedipine film solution, was pre-treated with a High Pressure Homogeniser.

3. Eudragit S was used as the polymer coat to provide an enteric coat with 0 drug release of up to 2-4 hours to the minicapsules, to target the drug release to the GIT and providing a pulsed release profile.

### EXAMPLE 7

Core Solution

| | | |
|---|---|---|
| Micronised Nifedipine | 500-1000 | grams |
| Gelatin | 500-3000 | grams |
| Sorbitol | 0-200 | grams |
| Purified Water | 4000-6000 | grams |

The minicapsules in Example 7 were manufactured according to Examples 1&2 and filled into suitable hard gelatin capsules to the required target strength.

### EXAMPLE 8

1. From Example 6 take a population of Immediate Release (IR) minicapsules.
2. Take a second population of Sustained Release (SR) minicapsules from Example 6
3. In the following ratio 5-25% Immediate Release (IR) and 75-95% Sustained Release (SR) minicapsules calculated by potency from Example 6 are blended using a suitable blender and encapsulated using suitable hard gelatin capsules into the target strengths.

### EXAMPLE 9

| Core Solution | | |
|---|---|---|
| Micronised Nimodipine USP/EP | 100-400 | grams |
| PEG 400 | 400-800 | grams |
| Median Solution | | |
| Vegetable Oil or Mineral Oil | 0-1000 | grams |
| Mucoadhesive Coating Solution | | |
| Ethycellulose | 5-100 | grams |
| PVP | 0.5-50 | grams |
| Castor Oil | 0-50 | grams |
| Magnesium Stearate | 0-50 | grams |
| Acetone | as required | |
| Isopropanol | as required | |
| Film Solution | | |
| Gelatin | 100-500 | grams |
| Sumatriptan | 0-100 | grams |
| Sorbitol | 0-50 | grams |
| Purified water | 500-3000 | grams |
| Polymer Coating Solution | | |
| Eudragit RL | 5% | w/w |
| Eudragit RS | 95% | w/w |
| Isopropyl Alcohol | as required | |
| Acetone | as required | |
| Talc | as required | |
| Minicapsule Diameter | 0.5-1.80 | mm |

The Nimodipine Multiparticulate Seamless Minicapsules were manufactured according to freund Industrial Co. Ltd U.S. Pat. No. 5,882,680 (Seamless Capsule and Method of Manufacturing Same), as described in the Summary of the Invention Section. This example allows for the inclusion of the active ingredient in the Film Solution (gelatine layer) as also described in the Summary of the Invention Section.

To apply a mucoadhesive coating, a coating solution of 7% ethylcellulose, 0.85% PVP and 1% magnesium stearate was dissolved in an isopropanol/acetone mixture. The solution was then sprayed coated onto the minicapsules using a suitable fluidised bed processor. Talc was used to prevent agglomeration of the minicapsules during the spray coating stage. The coated minicapsules were dried in an environmentally controlled drier at 40-50 deg.C for typically 12-24 hours.

In order to further coat the mucoadhesive coated seamless minicapsules, a coating solution of 6.25% Eudragit RL (5% w/w) and 6.25% Eudragit RS (95% w/w) dissolved in isopropyl alcohol/acetone mixture was sprayed coated onto the minicapsules using an automated fluidised bed processor. Talc was used to prevent agglomeration of the minicapsules during the spray coating stage. The coated minicapsules were further dried in an environmentally controlled drier at 40-50 deg.C for typically 12-24 hours.

The Nimodipine seamless minicapsules produced in Example 8 were the encapsulated using suitable hard gelatine capsules into typically 30/60/90/120 or 180 mg capsules or alternatively formats for rectal, vaginal or nasal administration.

Combination of Nimodipine and P-gp/P450 Inhibitors

### EXAMPLE 10

Nimodipine is metabolized through the cytochrome P450 system. By combining nimodipine with the cytochrome P450 inhibitor, carbamazepine (anticonvulsant), the clinical effectiveness of nimodipine may be increased (Clin Psychopharmacol., 1998, 18, 404-13). A nimodipine SEDDS (Self Emulsifying Drug Delivery System) formulation is prepared with polyoxyl hydrogenated castor oil. A formulation consisting of a modified vegetable oil (e.g., polyoxyl hydrogenated castor oil), a surfactant (e.g., TPGS), a co-solvent (e.g., propylene glycol) and a bile salt (e.g., sodium deoxycholate) is prepared by successive addition and mixing of each component. The nimodipine is then added to the formulation, which is thoroughly mixed to form a clear homogenous mixture. The carbamazepine is finally added and dissolved quickly under mild agitation. The nimodipine/carbamazepine pre-microemulsion concentrate is then formed into seamless microcapsules according to the methods described in U.S. Pat. Nos. 5,478,508 and 5,882,680 with an intermediate oil layer and an outer gelatin shell. The formulation for the intermediate oil layer and outer gelatin shell are the same as that outlined in Example 1.

Core Formulation

| Ingredients | % w/w |
|---|---|
| Nimodipine | 2.5-5 |
| Carbamazepine | 2.5 |
| Unconjugated deoxycholic acid | 5 |
| Fractionated oat oil | 30 |
| Cremophor EL or TPGS | 30 |
| PEG 400 | 30 |

### EXAMPLE 10a

Sustained release nimodipine/carbamazepine minicapsules may also be formulated by coating the seamless minicapsules (described in Example 10), with the rate-controlling polymer coat comprised Eudragit RS and Eudragit RL. The formulation and coating procedure for the Eudragit RL (5% w/w) and Eudragit RS (95% w/w) is the same as that outlined in Example 1.

### EXAMPLE 11

Another anticonvulsant, valproic acid, has also been shown to inhibit the presystemic oxidative metabolism of nimodipine, resulting in increased plasma concentrations of nimodipine when the two drugs are administered in combination (Drugs Aging, 1995, 6, 229-42).

A nimodipine/valproic acid SEDDS (Self Emulsifying Drug Delivery System) formulation is prepared with polyoxyl hydrogenated castor oil as described in Example 10 above, with the valproic acid replacing the carbamazepine in the formulation. The nimodipine/valproic minicapsules may be coated with a Eudragit RS and Eudragit RL polymer coat as described in Example 1.

### EXAMPLE 12

The antihistamine, cimetidine, has also been shown to produce an approximate doubling of the bioavailability of nimodipine, as a result of the known inhibitory effect of cimetidine on cytochrome P450 (Drugs Aging, 1995, 6, 229-42).

A nimodipine/cimetidine SEDDS (Self Emulsifying Drug Delivery System) formulation is prepared with polyoxyl hydrogenated castor oil as described in Example 10 above, with the cimetidine replacing the carbamazepine in the formulation. The nimodipine/cimetidine minicapsules may be coated with a Eudragit RS and Eudragit RL polymer coat as described in Example 10a.

Combinations of the Statins with Thiazide Diuretics, Beta Blockers, Ace Inhibitors, Folic Acid, Co-Enzme Q10 and Anticoagulants

### EXAMPLE 12

The risk of cardiovascular disease can be reduced by treating all the risk factors simultaneously. The risk factors include; LDL cholesterol (treated with simvastatin), blood pressure (treated with ACE inhibitor ramipril, the diuretic hydrochloridethiazide or the calcium channel blocker nimodipine), irregular heart beat (treated with the beta blocker atenolol), serum homocysteine (treated with folic acid), and platelet function (treated with the anticoagulant aspirin). For ease of administration and to simplify the CVD prevention treatment regime, it is preferable that some or all of the drugs mentioned above or formulated into a single dosage form. Obviously with the large number of actives involved, it can be difficult to achieve the drug loadings necessary using conventional dosage forms. The increased solubility conferred on the actives using the LEDDS technology can however be used to achieve the desired loadings.

Simvastatin, coenzyme Q10, ramipril, hydrochlorothiazide, nimodipine, and atenolol minicapusles are prepared by solubilising/suspending the actives in a suitable medium chain triglyceride (MCT) and forming into seamless microcapsules with an outer gelatin coating according to the methods described in U.S. Pat. Nos. 5,478,508 and 5,882,680. These minicapsules can also be formulated to include required concentrations of aspirin and folic acid either in the core or in the outer gelatin shell. In cases where the drug loadings required are particularly high, extra pharmaceutical active can also be incorporated into the shell. The formulation of the core formulations for the simvastatin, ramipril and co-enzyme Q10 below. The populations of minicapsule can also be coated with a sustained release polymer as described in Example 10a.

| Ingredients | % w/w |
|---|---|
| Simvastatin Core Formulation | |
| Simvastatin | 10-20 |
| MCT | 80-90 |
| Ramipril Core Formulation | |
| Ramipril | 10-20 |
| MCT | 80-90 |
| Co-enzyme Q10 Core Formulation | |
| Co-enzyme Q10 | 10-20 |
| MCT | |
| Hydrochlorothiazide Core Formulation | |
| Hydrochlorothiazide | 10-20 |
| MCT | 80-90 |
| Nimodipine Core Formulation | |
| Nimodipine | 10-20 |
| MCT | 80-90 |
| Atenolol Core Formulation | |
| Atenolol | 10-20 |
| MCT | 80-90 |
| Shell Solution | |
| Gelatin | 15-20 |
| Sorbitol/Glycerin | 1-5 |
| Purified Water | 70-80 |
| Folic acid | 1-5 |
| Aspirin | 1-5 |

In-Vitro/In-Vivo Correlations (IVIVC)

Computer generated simulations are used to predict the absorption of a drug when dosed in humans or animals. This software program can be used as a tool to theoretically predict the dissolution profile of a specific drug when designing a drug formulation. Thus, this prediction can theoretically predict the in-vivo profile of the specific drug.

The simulations and the software program is described below.

WinNonlin® Professional Edition Version 4.0.1 (Pharsight Corporation, Cary, USA) was utilized to undertake compartmental modelling and pharmacokinetic simulation of mean nimodipine plasma concentration versus time data. WinNonlin is a powerful program for the solution of nonlinear regression problems, constrained estimation problems, systems of differential equations, and mixtures of differential equations and functions. WinNonlin has quickly become the new industry standard in PK, PD, and Noncompartmental Analysis. The source data was derived from a study conducted by Gualano and co-workers at the Aster clinical research organization in Paris, France. Gualano and co-workers evaluated two formulations of nimodipine in a randomised four-way crossover replicate design study. The study subjects received both Test (two 30 mg film-coated Brainal® tablets) and Reference (two 30 mg film-coated Nimotop® tablets) treatments as a replicate administration of the two treatments according to a 4-period/2-sequence design (TRRT and RTTR), with a washout period of 7 days between each treatment. The study population was twenty-four normal healthy Caucasian male volunteers. The clinical part of the study was carried out using a replicate design in order to minimize the effects due to the high variability in the bioavailability of nimodipine and the high intra-individual variability.

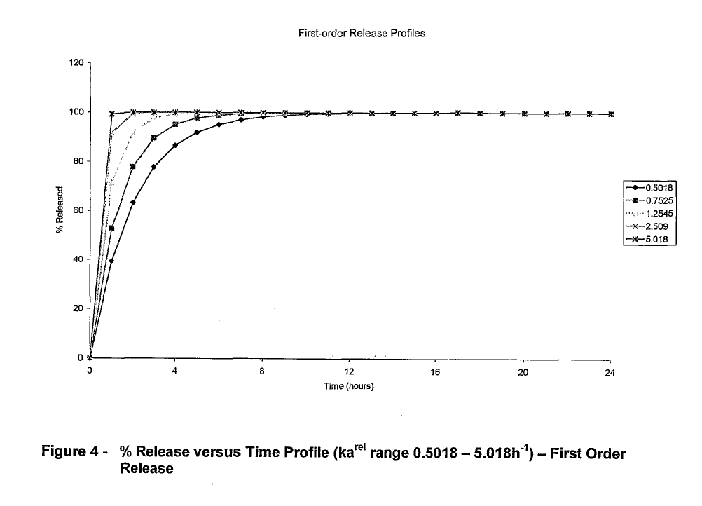

A number of different compartmental models (different number of compartments and with and without associated lag times) were fitted to the available source data. A two compartmental model with an associated lag time was selected as the most appropriate model to use for simulation purposes and initial estimates were determined. Following fitting of the mean IR data, first order release target profiles were generated for Ka^{rel} which resulted in 90% release of active over 1-5 hours. These target profiles are illustrated in the graph below. These ranges of ka values were deemed suitable and from these in vivo profiles were simulated. It is assumed that the release rate from the formulation (Ka^{rel}) is the predominant rate constant (compared to the absorption rate constant Ka). A ten-fold range of Ka values (0.5018-5.018 hrf⁻¹) were simulated to determine the optimal formulation approach which would maintain plasma concentration levels in excess of the proposed therapeutic target concentration of 30 ng/mL for the maximum duration. The impact of this range of Ka^{rel} values on the % release versus time profiles are presented in FIG. 4.

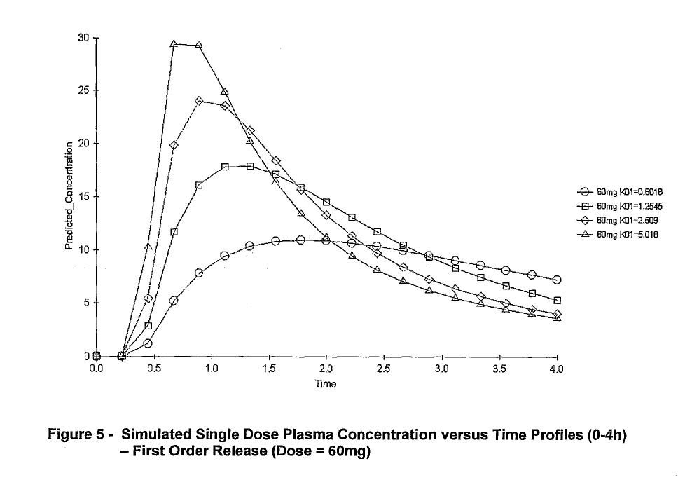

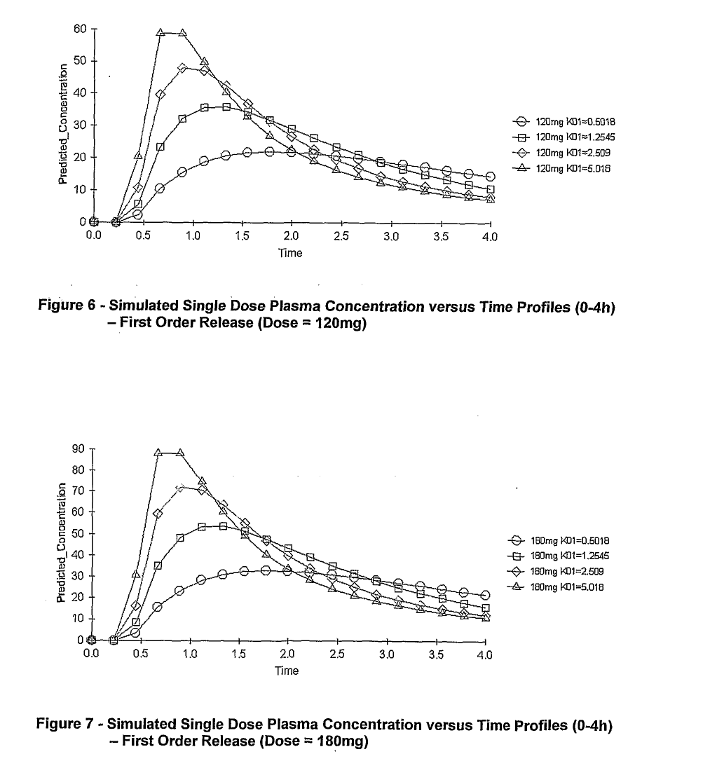

Using the initial estimates determined following the compartmental modelling of the source data, the following simulations were derived. FIGS. 5 to 8 simulate the administration of a 60 mg, 120 mg or 180 mg single dose of nimodipine over the ten-fold range of Ka values (0.5018-5.018 hr⁻¹) for a period of 4 hours post-dosing.

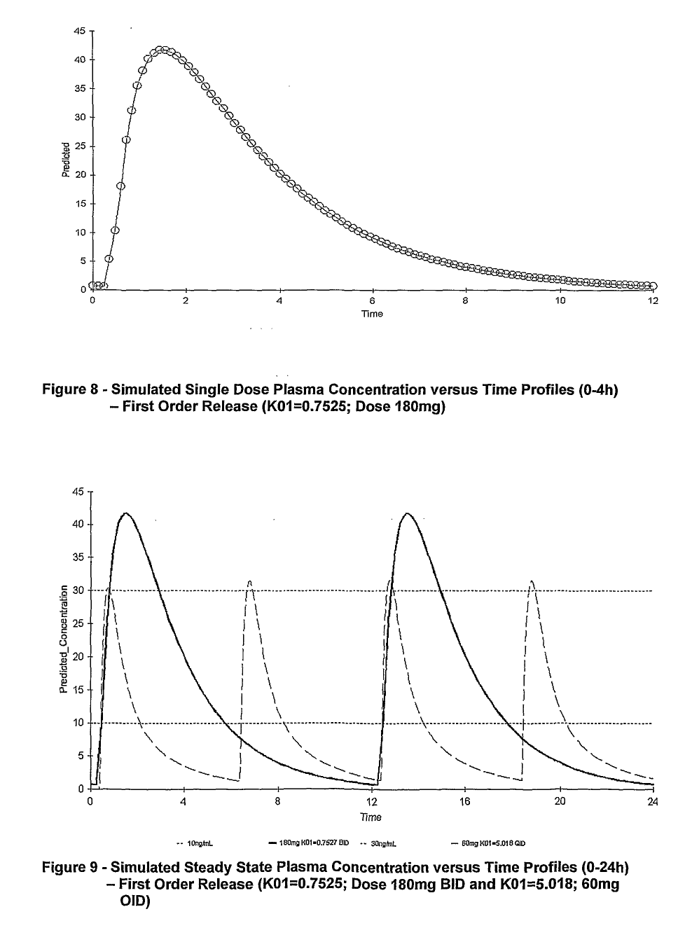

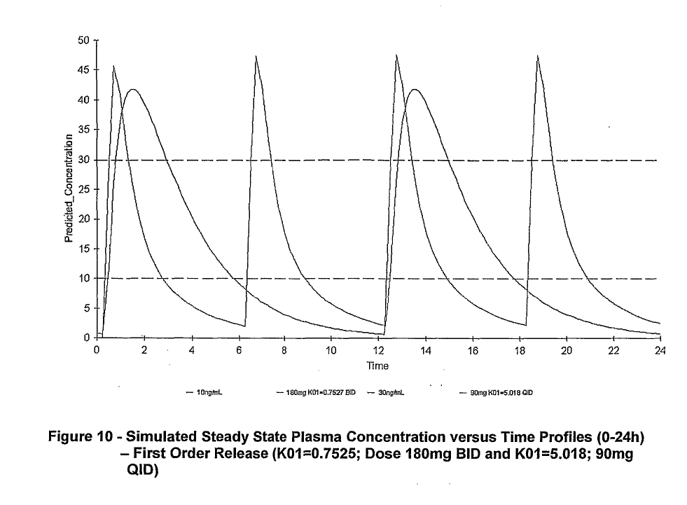

Upon review, an additional K01 value was simulated 0.7527 over a 12 h period as the optimal target profile was proposed as lying in the range between K01 0.5018 and 1.2545 hr⁻¹. The simulated single dose profile with this K01 at a dose level of 180 mg is presented in FIG. 8.

Steady state projections were then simulated with 180 mg nimodipine K01 of 0.7527 h⁻¹ following BID dosing compared to a reference of 60 mg nimodipine following QID dosing (K01=5.018).

Steady state projections were then simulated with 180 mg nimodipine K01 of 0.7527 h⁻¹ following BID dosing compared to a reference of 90 mg nimodipine following QID dosing (K01=5.018).

The simulated pharmacokinetic parameters following administration of 180 mg nimodipine (K01=0.7525 h⁻¹), 60 mg or 90 mg nimodipine (K01=5.018 h⁻¹).

FIG. 11

TABLE I
Simulated pharmacokinetic parameters -
First Order Release

| | | | | | | | Time > 10 |
|---|---|---|---|---|---|---|---|
| Treatment | AUCtau | Cmax | Cmin | Cavg | % Fl | Tmax | ng/mL |
| 180 mg | 162.32 | 41.74 | 0.68 | 13.51 | 303.51 | 1.45 | 5.29 |
| K01 | | | | | | | |
| 0.7527 h⁻¹ | | | | | | | |
| 90 mg | 82.00 | 47.39 | 2.21 | 13.66 | 330.54 | 0.75 | 2.58 |
| K01 | | | | | | | |
| 5.018 h⁻¹ | | | | | | | |
| 60 mg | 54.03 | 31.56 | 1.39 | 9.01 | 335.46 | 0.77 | 1.80 |
| K01 | | | | | | | |
| 5.018 h⁻¹ | | | | | | | |

The predicted steady state PK parameters show that the lowest degree of fluctuation occurs following administration of 180 mg K01=0.7525 h⁻¹ BID administration. The average concentrations were comparable across the range (9-14 ng/mL). The duration at which the plasma concentration levels remained in excess of 10 ug/L was 10.6 h following administration of 180 mg K01=0.7525 h⁻¹ BID, 7.2 h following administration of 60 mg K01=5.018 h⁻¹ and 10.3 h following administration of 80 mg K01=5.018 h⁻¹

Capsule-Minicapsule Cavity

When minicapsules are inserted into hard gelatine capsules, depending on the minicapsule size, a vacuum or interstitial or inter-minicapsule space exists. This space may be filled with various liquids, semi-liquids, powders or gases containing various active or inert entities, including drugs, excipients and preservatives. The filling material may be blended with minicapsules prior to filling hard gelatine capsules with the blended liquid, powder or gas.

Pill Format

Apart from insertion into hard gelatine capsules, minicapsules also may be blended with various excipients and/or actives prior to being pressed into tablet, pellet or pill formats that may further be coated with various controlled release polymers. Additionally, such pill formats may erode over time permitting controlled release of the minicapsules.

In a further embodiment, the tablet, pellet or pill format may be gastric retentive and swell in the stomach, preventing passage into the small intestine, thus releasing the minicapsule contents at various rates within the stomach.

The minicapsules may contain various additional ingredients and/or may be formulated as described in our two co-pending PCT applications filed Sep. 27, 2005 and entitled “Combination Products” and “Minicapsule Formulations”, the entire contents of which are herein incorporated by reference.

This invention is not limited to the embodiments hereinbefore described which may be varied in detail.

1-92. (canceled)

93: A pharmaceutical formulation comprising a plurality of seamless minicapsules having a diameter of from 0.5 mm to 5 mm, the minicapsules having an encapsulating medium, and the mincapsules containing a dihydropyrimidine as an active ingredient.

94: The formulation as claimed in claim 93 wherein the active ingredient is dispersed in the encapsulating medium.

95: The formulation as claimed in claim 93 wherein at least some of the minicapsules comprise a core containing the active ingredient.

96: The formulation as claimed in claim 95 wherein in at least some of the minicapsules the active ingredient in the core is solubilised in a pharmaceutically acceptable solvent and/or in a liquid phase.

97: The formulation as claimed in claim 93 wherein in at least some of the minicapsules the active ingredient is in a solid form and/or in a semi-solid form.

98: The formulation as claimed in claim 93 wherein the minicapsules have a diameter of from 0.5 mm to 3.0 mm.

99: The formulation as claimed in claim 93 wherein the minicapsules have a diameter of from 1.2 mm to 2.0 mm.

100: The formulation as claimed in claim 93 wherein the minicapsules have a diameter of from 1.4 mm to 1.8 mm.

101: The formulation as claimed in claim 93 wherein at least some of the minicapsules have at least one coating to control the time and/or location of the release of the active entity.

102: The formulation as claimed in claim 101 wherein at least one coating is an immediate release coating.

103: The formulation as claimed in claim 101 wherein at least one coating is a sustained release coating.

104: The formulation as claimed in claim 101 wherein the coating comprises a sustained release and an immediate release coating.

105: The formulation as claimed in claim 101 wherein at least one coating is an enteric coating.

106: The formulation as claimed in claims 101 wherein the rate-controlling coating is of a polymeric material.

107: The formulation as claimed in claim 101 wherein the rate-controlling coating comprises amino methacrylate polymeric material.

108: The formulation as claimed in claim 101 wherein the rate-controlling coating is an acrylate and/or methacrylate copolymer with quaternary ammonium.

109: The formulation as claimed in claim 101 wherein the coating comprises two copolymers, one of which is highly permeable and the other of which is poorly permeable.

110: The formulation as claimed in claim 109 wherein the weight ratio of highly permeable polymer to poorly permeable polymer is from 5:95 to 15:85.

111: The formulation as claimed in claim 110 wherein the ratio is from 10:90 to 15:85.

112: The formulation as claimed in claim 109 wherein the highly permeable copolymer is applied in the form of a polymer solution.

113: The formulation as claimed in claim 112 wherein the highly permeable copolymer is insoluble in water.

114: The formulation as claimed in claim 109 wherein the poorly permeable copolymer is applied in the form of a polymer solution.

115: The formulation as claimed in claim 114 wherein the poorly permeable copolymer is insoluble in water.

116: The formulation as claimed in claim 101 wherein the rate-controlling polymer coating contains methacrylate copolymer in the following ratio's 5:95; 10:90; 15:85 (w/w) as a mixture of Eudragit RL:Eudragit RS.

117: The formulation as claimed in claim 116 wherein the copolymer mixture comprises Eudragit RL 30D:Eudragit RS 30D.

118: The formulation as claimed in claim 116 wherein the copolymer mixture comprises Eudragit RL 12.5:Eudragit RS 12.5.

119: The formulation as claimed in claim 101 wherein the coating comprises an enteric coating of a methacrylate polymer.

120: The formulation as claimed in claim 119 wherein the enteric coating comprises Eudragit S 12.5 or Eudragit S100 providing 0 drug release in the stomach for up to 4 hours.

121: The formulation as claimed in claim 101 wherein at least some of the minicapsules are coated with an immediate release coating.

122: The formulation as claimed in claim 121 wherein the immediate release coating is applied to a rate controlling coating.

123: The formulation as claimed in claim 121 wherein the immediate release coating contains a pharmaceutically active ingredient.

124: The formulation as claimed in claim 122 wherein an immediate release pharmaceutically active ingredient solution is applied to a rate-controlling coating.

125: The formulation according to claim 93 wherein the active pharmaceutical ingredient is suspended or dissolved in the encapsulating medium of the seamless minicapsule.

126: The formulation as claimed in claim 93 wherein the encapsulating medium is of one or more of gelatine, agar, a polyethylene glycol, starch, casein, chitosan, soya bean protein, safflower protein, alginates, gellan gum, carrageenan, xanthan gum, phtalated gelatine, succinated gelatine, cellulosephtalate-acetate, oleoresin, polyvinylacetate, hydroxypropyl methyl cellulose, polymerisates of acrylic or methacrylic esters, polyvinylacetate-phtalate and combinations thereof.

127: The formulation according to claim 126 wherein the active ingredient is suspended or dissolved in the encapsulating medium.

128: The formulation as claimed in claim 93 wherein the active ingredient is micronised or nanonised.

129: The formulation as claimed in claim 128 wherein the active ingredient has a particle size of less than 100 microns.

130: The formulation as claimed in claim 93 wherein at least some of the minicapsules comprise a core containing micronised or nanonised active ingredient and the encapsulating medium contains micronised or nanonised active pharmaceutical ingredient suspended or dissolved in the encapsulating medium to enhance the potency of the seamless minicapsules.

131: The formulation as claimed in claim 93 wherein a permeability enhancing agent is suspended or dissolved in the encapsulating medium to enhance active bioavailability.

132: The formulation as claimed in claim 93 wherein at least some of the minicapsules comprise a buffer layer.

133: The formulation as claimed in claim 93 wherein at least some of the minicapsules are provided with or contain a bioadhesive such as a mucoadhesive.

134: The formulation as claimed in claim 133 wherein the bioadhesive comprises from 0% to 10% by weight of one or more of the following polymer classes:—polyacrylates; polyanhydrides; chitosans; carbopols; cellulose; methylcellulose; methylated deoxycellulose (m-doc™); lectins.

135: The formulation as claimed in claim 133 wherein the bioadhesive coating comprises from 0% to 10% by weight of one or more of the following thiolated or otherwise derivatised polymers:—polyacrylates; polyanhydrides; chitosans; carbopols; cellulose; methylcellulose; methylated deoxycellulose (m-doc™); lectins.

136: The formulation as claimed in claim 133 wherein the bioadhesive comprises a coating.

137: The formulation as claimed in claim 133 wherein the bioadhesive is incorporated into a part or layer of the minicapsule.

138: The formulation as claimed in claim 137 wherein the bioadhesive is incorporated into a rate-controlling layer.

139: The formulation as claimed in claim 137 wherein the bioadhesive is incorporated into the encapsulating medium.

140: The formulation as claimed in claim 93 wherein at least some of the minicapsules have a layer such as an outer layer which is divided into at least two parts.

141: The formulation as claimed in claim 140 wherein the parts are of the same composition.

142: The formulation as claimed in claim 140 wherein at least some of the parts have different compositions.

143: The formulation as claimed in claim 93 which comprises at least two populations of sustained release minicapsules.

144: The formulation as claimed in claim 143 wherein the populations have different in-vitro dissolution profiles.

145: A formulation of a dihydropyrimidine as the active ingredient comprising a plurality of seamless minicapsules having at least two populations selected from:—
a first minicapsule population in which the minicapsules comprise a core containing the active ingredient and an encapsulating medium, the minicapsules having a diameter of from 0.5 mm to 5 mm;
a second minicapsule population in which the minicapsules comprise a plurality of particles containing the active entity dispersed in an encapsulating medium, the minicapsules having a diameter of from 0.5 mm to 5 mm; and
a third micro or mini particles population in which the minicapsules comprise an inert core and at least one layer around the core, the layer containing the active ingredient.

146: The formulation as claimed in claim 93 wherein the dihydropyridine is nimodipine.

147: The formulation as claimed in claim 93 wherein the dihydropyridine is selected from felodipine, nicardipine nifedipine, istradipine, amlodipine and nisoldipine.

148: The formulation as claimed in claim 93 wherein at least some of the minicapsules comprise a core containing nimodipine.

149: The formulation as claimed in claim 93 wherein the formulation comprises an immediate release coating which contains nimodipine.

150: The formulation as claimed in claim 101 wherein the coating comprises a rate-controlling coating to achieve therapeutically effective plasma levels of the active ingredient over at least 12 hours in a human patient.

151: The formulation as claimed in claim 101 wherein the coating comprises a rate-controlling coating to achieve therapeutically effective plasma levels of the active ingredient over at least 24 hours in a human patient.

152: The formulation as claimed in claim 101 which provides a dissolution profile in a pre-determined media such that NMT 25% of the solubilised pharmaceutical active ingredient is released after 1 hour, NMT 40% after 4 hours, NMT 70% after 8 hours and 75 to 100% after 12 hours, alternatively the formulation provides a dissolution profile in pre-determined media such that 10 to 15% of the solubilised pharmaceutical active ingredient is released after 1 hour, about 15 to 30% is released after 4 hours, about 35 to 50% is released after 9 hours, about 45 to 65% is released after 12 hours and at least 80% is released after 24 hours.

153: The formulation as claimed in claim 93 wherein an enteric coated minicapsule is combined with two sustained release coated minicapsules to provide a pulsed release dissolution profile.

154: The formulation as claimed in claim 93 wherein greater than 80% (w/w by potency) of the formulation is comprised of sustained release minicapsules.

155: The formulation as claimed in claim 93 wherein the minicapsules provide extended residence times in the small intestine for a period of at least 5 hours, preferably at least 7 hours and more preferably in the 8-24 hours range to enable maximal bioactivity of the core active, locally or systemically.

156: The formulation as claimed in claim 93 wherein the minicapsules provide extended residence times in the nasal passage to enable maximal bioactivity of the core active agent, locally or systemically.

157: The formulation as claimed in claim 93 wherein the minicapsules provide extended residence times in the rectal passage to enable maximal bioactivity of the core active agent, locally or systemically.

158: The formulation as claimed in claim 93 wherein the minicapsules are capable of extended residence times in the vagina or intrauterine to enable maximal bioactivity of the core agent, locally or systemically.

159: The formulation according to claim 93 wherein the minicapsules are filled into hard gelatin capsules.

160: The formulation according to claim 93 wherein the minicapsules are filled into a sachet.

161: The formulation according to claim 93 wherein the minicapsules are suspended in oil as a lubricant.

162: The formulation according to claim 93 wherein the minicapsules are contained within a wide gauge syringe that is compatible with tube delivery.

163: The formulation according to claim 93 wherein the minicapsules are in the form of a sprinkle.

164: The formulation according to claim 93 wherein the minicapsules are formulated as a suppository for rectal or vaginal administration.

165: The formulation according to claim 93 wherein the minicapsules are formulated for nasal administration.

166: The formulation as claimed in claim 93 wherein the formulation contains at least one further active entity.

167: The formulation as claimed in claim 166 wherein the further active entity is a P-gp/P450 inhibitor.

168: The formulation as claimed in claim 166 wherein the further active entity is carbamazepine.

169: The formulation as claimed in claim 166 wherein the further active entity is valproic acid.

170: The formulation as claimed in claim 166 wherein the further active entity is cimetidine.

171: The formulation as claimed in claim 166 wherein the further active entity is a tryptan.

172: The formulation as claimed in claim 166 wherein the further active entity is sumatriptan.

173: The formulation as claimed in claim 166 for the treatment of Alzheimers disease wherein the further active entity comprises a cholinesterase inhibitor (such as donepezil, rivastigmine, galantamine) and one or more from the following classes: vitamins, statins, estrogen, nootrophic agents, ginkgo biloba, anti-inflammatory agents, anti-depressants, anti-psychotics, and mood stabilizers.

174: The formulation as claimed in claim 166 wherein the further active entity is selected from one or more of a statin, a thiazidediuretic, a beta blocker, an ACE inhibitor, folic acid, co-enzyme Q10, and an anticoagulant.

175: The formulation as claimed in claim 166 wherein the further active entity is present in a seamless minicapsule.

176: The formulation as claimed in claim 166 wherein the further active entity is present in at least some of the seamless minicapsules.

177: The formulation as claimed in claim 93 comprising a capsule containing a plurality of minicapsules.

178: The formulation as claimed in claim 177 wherein the capsule contains another entity.

179: The formulation as claimed in claim 178 wherein the other entity is in a liquid, powder, semi-solid, solid or gaseous form.

180: The formulation as claimed in claim 172 wherein the other entity comprises an active entity.

181: The formulation as claimed in claim 93 comprising a tablet or pellet containing a plurality of minicapsules.

182: The formulation as claimed in claim 181 wherein the tablet or pellet contains another entity.

183: The formulation as claimed in claim 182 wherein the other entity is an active entity.
